(19) United States
(12) Patent Application Publication
(10) Pub. No.: US 2004/0058023 A1
(43) Pub. Date: Mar. 25, 2004

(54) Skin external preparation containing pine pollen or pine pollen extract

(76) Inventors: Joon Whan Chang, Seoul (KR); Bum Roh Lee, Seoul (KR)

Correspondence Address: DARBY & DARBY P.C., P. O. BOX 5257, NEW YORK, NY, 10150-5257, US

(21) Appl. No.: 10/399,383

(22) PCT Filed: Oct. 17, 2001
(86) PCT No.: PCT/KR01/01743

(30) Foreign Application Priority Data
Oct. 20, 2000 (KR) 2000/62071

(51) Int. Cl. A61K 7/06; A61K 35/78

(52) U.S. Cl. 424/770; 424/74

(57) ABSTRACT

The present invention relates to a skin external preparation containing pine pollen or pine pollen extract. The pine pollen or pine pollen extract inhibits a growth of the acne bacterium Propionibacterium acnes and exhibits an effect of reducing comedos, and thus is effective for the prevention and treatment of acne. Preferably, the pine pollen or pine pollen extract is contained in an amount ranging from 0.05% by weight to 50% by weight, based on the total weight of the skin external preparation.

### TECHNICAL FIELD

The present invention relates generally to a skin external preparation, and more particularly to a skin external preparation containing pine pollen or pine pollen extract, which has an excellent antibacterial activity against the acne bacterium Propionibacterium acnes and a comedo reducing effect.

### BACKGROUND ART

Acne is called acne vulgaris and is one of skin characteristics of youths. Generally, factors that cause acne are not clearly identified; it is however known that they vary depending on humans, and an interaction of several factors causes an attack of acne. The first of the main factors that cause acne is excessive sebum produced by active sebum producing action of testosterone. Second, excessive sebum produced by clogging of a skin pore with a hyperkeratinized hair follicle is accumulated in the skin pore to promote formation of comedos, thereby increasing the acne bacterium Propionibacterium acnes. Third, the acne bacterium Propionibacterium acnes secretes a lipase enzyme that hydrolyzes triglyceride that is one of excessive sebum components produced by hypertrophic growth of sebaceous glands and hyperkeratinization of hair-follicle pores. This hydrolysis produces free fatty acids.

The free fatty acids produced as described above act on the skin epithelium, so as to produce various enzymes and also rupture the follicular wall to cause an inflammatory reaction in connective tissues around the hair follicle. Depending upon the degree of inflammation, papules, pustules, cysts, nodules, and in the worst cases, scars may develop.

It has been known up to now that, where antibiotics such as tetracycline and the like as antibacterial agents against the acne bacterium Propionibacterium acnes are used in preventing and treating acne, they tend to cause adverse side effects such as an appearance of Propionibacterium acnes-tolerant bacteria or the photosensitization. It is also known that salicylic acid or retinoic acid preparations mainly effective for the removal of keratin, as well as benzoyl peroxide preparations or topical antibiotics and the like having an inhibitory activity against suppurative bacteria, cause much adverse side effects and also do not exhibit an effect of completely curing acne.

The salicylic acid preparations cannot be used for purulent cutitis and can cause skin flare and erythema. The benzoyl peroxide preparations can cause allergic cutitis and erythema. It is reported that the retinoic acid preparations are able to cause flare and edema and develop hyperpigmentation symptoms when they are continuously used. The topical antibiotics are disadvantageous in that they do not have a fixed safety for a pregnant or nursing woman and cannot be used for an antibiotic hypersensitive patient. Moreover, there were recently conducted much studies to find substances having an inhibitory activity against 5 alpha-reductase, and as a result, several substances were developed. It is known that the developed substances, however, have an insufficient effect and are difficult to put to practical use.

### DISCLOSURE OF THE INVENTION

The present inventors have carried out extensive studies on natural products for a lengthy period of time to solve the problems described above, and consequently found that pine pollen or its extract exhibits an excellent effect for the treatment of acne and a high safety without causing any side effects. As a result of continued studies on this finding, it was found that pine pollen or its extract exhibits an excellent antibacterial activity against the acne bacterium Propionibacterium acnes, as well as a comedo reducing effect and an excellent effect for the treatment of acne, and at the same time can reduce the side effects and drawbacks of the acne-treating agents according to the prior art. Based on this discovery, the present invention was achieved.

It is therefore an object of the present invention to provide a skin external preparation, which has an excellent antibacterial activity against the acne bacterium Propionibacterium acnes and a comedo reducing effect and thus is effective in preventing and treating acne, while being capable of reducing the side effects and drawbacks of the acne-treating agents according to the prior art.

The present invention provides a skin external preparation containing pine pollen or pine pollen extract in an amount effective to prevent and treat acne. The pine pollen or pine pollen extract is preferably contained in the amount ranging from about 0.05% by weight to about 50% by weight, more preferably about 1% by weight to about 20% by weight, and most preferably about 2% by weight to about 10% by weight, based on the total weight of the skin external preparation. The skin external preparation containing pine pollen or its extract is used in the form of a treating agent for pharmaceutical application, a toilet water, a cream, a pack, a lotion, a foundation, a sun screen, a baby powder, a soap, a cleansing foam, a hair shampoo, and a body shampoo.

The pine pollen used in the practice of the present invention has been traditionally known as being effective in preventing palsy and treating chronic diarrhea, boil and skin eczema. Also, the pine pollen has been used for the treatment of wound as it has a hemostatic action.

The pine pollen extract used in the practice of the present invention was found to exhibit an excellent antibacterial activity against the acne bacterium Propionibacterium acnes and show an antibacterial effect regardless of physical properties of the skin external preparation when being applied for the skin external preparation. Moreover, it was found from clinical tests that the pine pollen extract has an acne treating effect and does not produce any side effects on the skin even when it is used repeatedly for an extended period of time.

Where the pine pollen itself or pine pollen extract used in the practice of the present invention is applied for the skin external preparation, it is preferably contained in an amount ranging from about 0.05% by weight to about 50% by weight relative to the total weight of the skin external preparation. This is because an acne treating effect and an antibacterial effect are insufficient if the pine pollen or the pine pollen extract is used in the amount less than 0.05% by weight. On the other hand, if it is used in the amount more than 50% by weight, a synergistic effect of the treatment is insufficient and a phase stability of the skin external preparation is inferior. More preferably, the pine pollen or the pine pollen extract is contained at the amount of about 1% by weight to about 20% by weight, and most preferably about 2% by weight to about 10% by weight.

Examples of bases that can be used in the skin external preparation of the present invention include cleansing agents for the cleansing of a human body, such as conventional soaps and cleansing foams, etc., and cosmetics, such as creams, lotions and foundations, etc. The bases may be present in any state of liquid, solid, cream and paste states.

### BEST MODE FOR CARRYING OUT THE INVENTION

The present invention will hereinafter be described in further detail by examples. It should however be borne in mind that the present invention is not limited to or by the examples.

### EXAMPLE 1

Evaluation of Antibacterial Activity of Pine Pollen Extract against Propionibacterium Acnes

A test for evaluating an antibacterial activity of pine pollen extract was carried out by adding the pine pollen extract of a given concentration to the acne bacterium Propionibacterium acnes ATCC 6919 cultured in a liquid medium, culturing the resulting mixture in an anaerobic cultivator, and then measuring a minimum growth inhibitory concentration (MIC) of the extract against the acne bacterium Propionibacterium acnes. The test procedure is as follows.

1. Culture Method

The acne bacterium Propionibacterium acnes ATCC 6919, a typical acne bacterium obtained from American Type Culture Collection, 10801 University Boulevard, Manassas, Va. 20110-2209, USA, is cultured in a reinforced clostridial medium for three days at 37° C., and then subcultured until a viability becomes better. The culture method is as follows.

a) Liquid Culture

10 ml of a reinforced clostridial medium is put into a screw cap tube and sterilized at 121° C. for 20 minutes. After sterilization, 0.5 ml of liquid paraffin, which was separately sterilized, is dropped onto the liquid medium immediately to prevent the liquid medium from being in contact with oxygen. As the medium gets cold, it is inoculated with the bacterium and cultured at 37° C. for 72 hours.

b) Solid Culture

Agar is added to a reinforced clostridial medium at 1.0% to prepare a solid medium for the storage of a strain. The solid medium is introduced into a screw cap tube at the amount of 10 ml and sterilized at 121° C. for 20 minutes. As the medium gets cold, it is inoculated with the bacterium using a platinum loop and stab cultured at 37° C. for 72 hours.

c) Plate Culture

A medium for plate culture is the same as the medium for solid culture. An agar medium is poured onto a plate and cooled. As the plate medium completely gets cold and hard, the plate medium is inoculated with the bacterium and introduced into an anaerobic jar. After 10 ml of water is poured into a Gas Pak of the anaerobic jar, the anaerobic jar is closed with a cap and the bacterium is cultured at 37° C. for 72 hours.

2. Preparing Method of Pine Pollen Extract

a) 100 g of pine pollen is introduced into an extractor, to which 2 liters of ethanol is then added and mixed well. Thereafter, the resulting mixture is extracted at 60° C. for three days.

b) An extract resulted from the extraction is filtered through Wattman Filter Paper No. 2, a filter, and a filtrate passed through the filter is taken and a residue caught by the filter is discarded.

c) The filtrate is introduced into a concentrating flask and then concentrated in vacuum to remove all ethanol. Also, the remaining water is removed by concentration.

d) The concentrated extract liquid of pine pollen is frozen in a deep freezer at −80° C. and freeze-dried to obtain 20 g of the pine pollen extract as a powder.

3. Test for Growth Inhibitory Effect of Pine Pollen Extract Against Propionibacterium Acnes ATCC 6919

a) 1 ml of a culture solution of the bacterium Propionibacterium acnes ATCC 6919, which was sufficiently subcultured, is inoculated to 9 ml of a reinforced clostridial medium and mixed well, thereby obtaining a 10-fold dilution.

b) The above step (a) is repeated in such a manner that 10⁻¹-10⁻⁵ times dilutions are obtained.

c) 10 g of pine pollen extract produced by freeze-drying is sufficiently dissolved in 10 ml of 50% ethanol, and serially two-fold diluted to produce a plurality of pine pollen extract solutions having various extract concentrations. These solutions are sterile-filtered using a 0.2 μm membrane filter.

d) A reinforced clostridial agar medium is sterilized and then cooled to 50° C. To the agar medium, the respective sterile-filtered extract solutions from the step c) are added in such a manner that a final concentration of the extracts is in the range of 10 mg/ml to 0.1 mg/ml. Then, the resulting mixtures are uniformly mixed well.

e) 1 ml of the respective diluted culture solutions from the above step b) are dispensed into a Petri dish. The respective agar media having the different extract concentration are dispensed in the Petri dish, shaken for uniform mixing, and then left to stand to be cured.

f) As the agar media are cured, they are placed into an anaerobic jar which is then equipped with a Gas-Pak. After the agar media are incubated at 37° C. for three days, the resulting colony number is measured and compared to a control group. Then, a percent inhibition of the acne bacterium Propionibacterium acnes by the pine pollen extract is calculated. Measured results of the colony number and the percent inhibition are shown in Table 2 below.

From evaluation of the bactericidal activity against the acne bacterium Propionibacterium acnes, it was found that the pine pollen extract of the present invention begins to show an antibacterial activity at a very low concentration of about 0.06% and completely kill the acne bacterium Propionibacterium acnes at a concentration of 0.1% or above, as shown in Table 1 below. It could therefore be found that the pine pollen extract has a very excellent antibacterial effect against the acne bacterium Propionibacterium acnes.

TABLE 1
Measured results of minimum inhibitory concentration
(MIC) against Propionibacterium acnes ATCC 6919

| Extract concentration (%) | Growth of acne bacterium |
|---|---|
| 1.0 | − |
| 0.5 | − |
| 0.25 | − |
| 0.125 | − |
| 0.063 | ± |
| 0.031 | + |
| 0.016 | + |

−: no
+: yes

TABLE 2
Test results of antibacterial effect against
Propionibacterium acnes

| | Control | 10 | 5 | 2.5 | 1.25 | 0.63 | 0.32 | 0.16 | 0.08 |
|---|---|---|---|---|---|---|---|---|---|
| Extract conc. | group | mg/ml | mg/ml | mg/ml | mg/ml | mg/ml | mg/ml | mg/ml | mg/ml |
| CFU | 417 | — | — | — | — | 2 | 50 | 123 | 265 |
| (Number/ml) | | | | | | | | | |
| Inhibition (%) | — | 100 | 100 | 100 | 100 | 99.52 | 88.01 | 70.50 | 36.45 |

### EXAMPLE 2

Comedo reducing effect of pine pollen extract In a test for measuring a reduction in comedos, New Zealand white rabbits were used as test animals. For this test, healthy male rabbits (about 4 months old, body weight of 2-3 kg) were purchased and subjected to an acclimation process for one week. Among the acclimated animals, only animals that look healthy when observed with the naked eye were used in the test. As a test substance for the comedo reduction test, a 1% solution of a concentrated pine pollen extract dissolved in ethanol was used. The test was carried out as follows.

1 ml of isopropyl myristate (IPM), a comedo inducer, was applied to both ears of thirty rabbits for seven days, one time each day, so as to induce comedo. Twenty-five animals in which a typical comedo had been formed were selected and divided into five test groups, each of which consists of five animals.

In the test for measuring a comedo reducing effect, 1 ml of the pine pollen extract-containing test substance prepared as described above was administrated to a right ear having the comedo formed thereon. Then, the administrated test substance was uniformly applied with a swab. 1 ml of ethanol used as a base was administrated to the left ear one time each day and then uniformly applied with a swab about ten times. The left ear was compared with the right ear.

Healing of comedo was progressed for two weeks, during which the test substance was administrated while a lesion progress degree of comedo and a comedo reducing effect were evaluated. After the test was ended, the test animal groups were euthanized, both ears of the respective animals were cut off, and a basal tissue was cut to have a size of about 2.5 cm×1.5 cm. The tissue was immersed in water of 50° C. for two minutes and then taken out from water. After peeling off an epidermis, the tissue was placed on a slide glass in such a manner that the inside face of the tissue faces upward. After drying, the tissue was observed with a stereoscopic microscope, and the number and area of comedo in the tissue were calculated with a picture analyzer. Results are shown in Table 3 below. From Table 3, it could be found that the area of comedos in the treated groups was remarkably reduced.

TABLE 3
Percent comedo reduction at two weeks after application
of pine pollen extract

| | Test groups | | | | |
|---|---|---|---|---|---|
| | Group 1 | Group 2 | Group 3 | Group 4 | Group 5 |
| A: comedo area in | 0.28 ± | 0.30 ± | 0.32 ± | 0.31 ± | 0.29 ± |
| left ear/number (cm²) | 0.01 | 0.01 | 0.02 | 0.02 | 0.01 |
| B: comedo area in | 0.07 ± | 0.08 ± | 0.10 ± | 0.09 ± | 0.07 ± |
| right ear/number | 0.01 | 0.01 | 0.02 | 0.02 | 0.01 |
| (cm²) | | | | | |
| Comedo reduction | 75 | 73.3 | 68.8 | 71 | 75.9 |
| (%)* | | | | | |

*Percent reduction in comedo = (A − B)/A × 100

### EXAMPLE 3

Antibacterial Activity of Pine Pollen Extract-Containing Pack Preparation Against Propionibacterium Acnes

To verify an antibacterial effect of the pine pollen extract described above against the acne bacterium Propionibacterium acnes when the pine pollen extract is applied to pack preparations, the pack preparations were prepared according to a conventional method and measured for their antibacterial effect against the acne bacterium. The pack preparations have a composition given in Table 4 below.

TABLE 4
Composition of pack preparations containing pine pollen
extract (% by weight)

| | Content (% by weight) | | | | | |
|---|---|---|---|---|---|---|
| | Control | Test | Test | Test | Test | Test |
| Component | group | group 1 | group 2 | group 3 | group 4 | group 5 |
| Kaolin | 15.0 | 15.0 | 15.0 | 15.0 | 15.0 | 15.0 |
| Glycerine | 5.0 | 5.0 | 5.0 | 5.0 | 5.0 | 5.0 |
| Ethanol | 5.0 | 5.0 | 5.0 | 5.0 | 5.0 | 5.0 |
| Bentonite | 4.0 | 4.0 | 4.0 | 4.0 | 4.0 | 4.0 |
| Glyceryl | 3.0 | 3.0 | 3.0 | 3.0 | 3.0 | 3.0 |
| monostearate | | | | | | |
| Cetostearyl | 3.0 | 3.0 | 3.0 | 2.0 | 2.0 | 2.0 |
| alcohol | | | | | | |
| Polyoxy- | 2.0 | 2.0 | 2.0 | 2.0 | 2.0 | 2.0 |
| ethylene | | | | | | |
| monostearic | | | | | | |
| ester | | | | | | |
| Titanium oxide | 2.0 | 2.0 | 2.0 | 2.0 | 2.0 | 2.0 |
| Pine pollen | — | 1.0 | 5.0 | 10.0 | 15.0 | 20.0 |
| oxide | | | | | | |
| Purified water | 61.0 | 60.0 | 56.0 | 51.0 | 46.0 | 41.0 |

To measure an antibacterial effect of the pack preparations prepared as described above against Propionibacterium acnes ATCC 6919, 100 μl of a sufficiently activated culture solution of the acne bacterium was 10-fold, 100-fold and 1,000-fold diluted, respectively, and inoculated to a Petri dish containing a suitable medium. The culture solution was then uniformly plated, incubated in an anaerobic incubator for three days, and measured for a grown colony (A). At the same time, 100 μl of a 5% solution of the respective test groups shown in Table 4 is inoculated to a Petri dish together with the dilution of the acne bacterium culture solution, uniformly plated, incubated in an anaerobic incubator for three days, and measured for a grown colony (B) and thus an antibacterial effect. The test groups were determined as being effective when they exhibit a sterilizing rate of 90% or above. The sterilizing rate was calculated according to the following equation:

Sterilizing rate=[(A−B)/A]×100

The antibacterial effect of the pack preparations is shown in Table 5 below.

TABLE 5
Antibacterial effect of pine pollen extract-containing
pack preparations against acne bacterium

| | Test groups | | | | | |
|---|---|---|---|---|---|---|
| | Control | Test | Test | Test | Test | Test |
| Colony | group | group 1 | group 2 | group 3 | group 4 | group 5 |
| A (CFU/ml) | 1533 | 1416 | 1629 | 1473 | 596 | 1509 |
| B (CFU/ml) | 1050 | 252 | 69 | 12 | 11 | 6 |
| Sterilizing rate | 31.5 | 82.2 | 95.8 | 99.2 | 99.3 | 99.6 |
| (%) | | | | | | |

From Table 5, it could be found that the antibacterial effect against the acne bacterium of the pack preparations containing the pine pollen extract is excellent, in comparison with the control group. It could be also found that the pack preparations containing the pine pollen extract at 5% or above exhibit a sterilizing rate of 95% or above and the pack preparations containing the pine pollen extract at 10% or above show a sterilizing rate of 99% or above. It could be thus found that an increase in concentration of the pine pollen extract does not result in a significant change in the antibacterial effect.

### EXAMPLE 4

Comedo Reducing Effect of Pine Pollen Extract-Containing Pack Preparations for Prevention and Treatment of Acne

To test a comedo reducing effect of pine pollen extract-containing pack preparations suitable for the prevention and treatment of acne, the pack preparations for clinical tests prepared in Example 3 were used as test pack preparations, and New Zealand white rabbits were used as test animals. For the test, healthy male rabbits (about 4 months old, body weight of 2-3 kg) were purchased and subjected to an acclimation process for one week. Among the acclimated animals, only animals that look healthy when observed with the naked eye were used in the test. The test was carried out as follows.

1 ml of isopropyl myristate (IPM), a comedo inducer, was applied to both ears of thirty rabbits for seven days, one time every day, so as to induce comedo. Twenty-five animals in which a typical comedo had been formed were selected and divided into five test groups, each of which consists of five animals.

In a test for measuring a comedo reducing effect, 2 g of the respective pine pollen extract-containing pack preparations prepared in Example 3 was administrated to a right ear having the comedo formed thereon, a time each day. Then, the administrated pack preparations were uniformly applied with a swab. 2 g of a pine pollen extract-not containing pack preparation, a control group, was administrated to the left ear one time each day and then uniformly applied with a swab about ten times. The left ear was compared with the right ear.

Healing of comedo was progressed for two weeks, and the pack preparations were sufficiently washed out or removed after applied. While administrating the pack preparations, a lesion progress degree of comedo and a comedo reducing effect were evaluated. After the test was ended, the test animal groups were euthanized and measured for the comedo reducing effect according to the same procedure as described in Example 2. Results are shown in Table 6 below. From Table 6, it could be found that an area of comedos in the treated groups was remarkably reduced.

### EXAMPLE 5

Clinical Test of Pack Preparation Containing Pine Pollen Extract

A clinical test for preventing and treating acne was performed using the pack preparations prepared in Example 3. Also, this clinical test was performed on volunteers consisting of thirty men and thirty women in their middle teens to middle thirties who suffer from acne and have skin conditions belonging to skin grades 3-9 in Table 7. For the test, the pack preparation of the respective test groups (including control group) was selected for every ten volunteers. The selected pack preparation was applied to the volunteers one time each day for one month. Results of the clinical test are shown in Table 8.

TABLE 7
Skin grades by observation with the naked eye

| Grades | Skin conditions |
|---|---|
| 0 | Normal |
| 1 | State where comedo is very small in number and size, and |
| | inflammation is not developed. |
| 2 | State where comedo is intermediate in number size, and |
| | inflammation is not developed. |
| 3 | State where small inflammatory papules are partially present. |
| 4 | State where small inflammatory papules are present over a wide |
| | area. |
| 5 | State where large inflammatory papules are present over a wide |
| | area. |
| 6 | State where pustules having pus formed therein are partially |
| | developed. |
| 7 | State where a large number of pustules are present. |
| 8 | State where severe nodular cysts are partially present. |
| 9 | State where severe cysts are present all over a face |

TABLE 8
Results of clinical test for pack preparations
containing pine pollen extract (persons)

| | Test groups | | | | | |
|---|---|---|---|---|---|---|
| | Control | Test | Test | Test | Test | Test |
| Effect | group | group 1 | group 2 | group 3 | group 4 | group 5 |
| Excellent | 0 | 0 | 6 | 9 | 9 | 10 |
| Good | 1 | 4 | 3 | 0 | 1 | 0 |
| Unchanged | 7 | 6 | 1 | 1 | 0 | 0 |
| Worse | 2 | 0 | 0 | 0 | 0 | 0 |
| Side effect | 0 | 0 | 0 | 0 | 0 | 0 |
| Total | 10 | 10 | 10 | 10 | 10 | 10 |

Table 8 as above shows that the pack preparations containing pine pollen extract (test groups 1-5) were far superior in the acne treating effect, as comparison to the control group. The pack preparation containing pine pollen extract at the amount of 1% (test group 1) was somewhat effective, in comparison with to the control group. Also, the pack preparations containing pine pollen extract at the amount of 5% or above (test groups 2-5) have exhibited a very excellent treating effect and have not caused any side effects. These results of the clinical test are based on an observation with the naked eye.

### EXAMPLE 6

In order to carry out clinical tests for soap, cream, toilet water, lotion and foundation preparations, which respectively contain pine pollen extract, skin external preparations having compositions given in Table 9-13 were prepared according to a conventional method.

TABLE 9
Composition of soap preparation containing pine pollen extract

| Components | Content (% by weight) |
|---|---|
| Soap base | 93.77 |
| Perfume | 1.2 |
| Pigment | 0.01 |
| Antioxidant | 0.02 |
| Pine pollen extract | 5.0 |

TABLE 10
Composition of cream preparation containing pine pollen extract

| Components | Content (% by weight) |
|---|---|
| Macademia oil | 2.0 |
| Squalan | 3.0 |
| Stearic acid | 7.0 |
| Vaseline | 3.0 |
| 1,3-butylene glycol | 7.0 |
| Polyoxyethylene sorbitan monostearate | 1.3 |
| Self-emulsifying glycerin monostearate | 2.7 |
| Sodium ethylenediamine tetraacetate | 0.05 |
| Glycerin | 4.0 |
| Perfume | 0.25 |
| Preservative | Proper quantity |
| Antioxidant | Proper quantity |
| Pine pollen extract | 10.0 |
| Purified water | Balance |

TABLE 11
Composition of toilet water preparation containing pine
pollen extract

| Components | Content (% by weight) |
|---|---|
| Citric acid | 0.1 |
| Salicylic acid | 0.5 |
| 1,3-butyleneglycol | 5.0 |
| Polyoxyethylene oleyl ether | 0.8 |
| Ethanol | 30.0 |
| Perfume | 0.15 |
| Preservative | Proper quantity |
| Pine pollen extract | 2.0 |
| Purified water | Balance |

TABLE 12
Composition of lotion preparation containing pine pollen extract

| Components | Content (% by weight) |
|---|---|
| Stearic acid | 0.5 |
| Ethanol | 10.0 |
| Vaseline | 1.0 |
| Squalan | 2.5 |
| Liquid paraffin | 6.0 |
| Polyoxyethylene sorbitan monooleate | 1.5 |
| 1,3-butylene glycol | 8.0 |
| Sodium ethylene diamine tetraacetate | 0.05 |
| Triethanolamine | 0.8 |
| Perfume | 0.2 |
| Preservative | Proper quantity |
| Antioxidant | Proper quantity |
| Pine pollen extract | 4.0 |
| Purified water | Balance |

TABLE 13
Composition of foundation preparation containing pine
pollen extract

| Components | Content (% by weight) |
|---|---|
| Stearic acid | 2.0 |
| 1,3-bytylene glycol | 2.0 |
| Cetylstearyl alcohol | 0.5 |
| Liquid lanolin | 1.5 |
| Liquid paraffin | 3.5 |
| Myristate isopropyl | 4.5 |
| Sodium carboxyethylcellulose | 0.3 |
| Bentonite | 1.0 |
| Glycerin | 1.0 |
| Titanium dioxide | 5.0 |
| Talc | 6.0 |
| Pigment | Proper quantity |
| Perfume | 0.15 |
| Preservative | Proper quantity |
| Antioxidant | Proper quantity |
| Pine pollen extract | 2.0 |
| Purified water | Balance |

Using the respective preparations having the composition given in Tables 9-13, the clinical test for preventing and treating acne was performed on volunteers consisting of five men and five women who respectively have a skin condition corresponding to skin grades 3-9 in Table 7. The foundation preparation was used for only women. The soap, cream, lotion and toilet water preparations were used twice each day for one month and the foundation preparation was used one time each day for one month, and results from the use of the preparations were then evaluated. In evaluating the results, the skin condition of the respective volunteers was graded by observation with the naked eye according to the skin grades given in Table 7 before and after the clinical test. Where the skin conditions of the volunteers are improved by three grades or above after the clinical test, it was rated as excellent. Where the skin condition was improved by one or two grades after the clinical test, it was rated as good. The evaluation results are given in Table 14 below.

TABLE 14
Results of clinical test for various preparations
containing pine pollen

| Effects | Persons |
|---|---|
| Excellent | 7 |
| Good | 2 |
| Unchanged | 1 |
| Worse | 0 |
| Side effect | 0 |
| Total | 10 |

### EXAMPLE 7

To perform a clinical test for the acne preventing and treating effects of pine pollen itself, pine pollen and Vaseline were mixed at a ratio of 1:2 to prepare a cream preparation. Using this cream preparation, the clinical test was performed on volunteers consisting of five men and five women who have a skin condition corresponding to skin grades 3-9 in Table 7. The cream preparation was applied to the respective volunteers a time each day for one month, and results from the use of the preparation were then evaluated. In addition, the cream preparation was used only at night, because it is not preferable that the cream preparation is used outdoors, because of an intrinsic color of pine pollen and a property and state of the preparation. In evaluating the results, the skin conditions of the respective volunteers are graded through observations with the naked eye according to the skin grades given in Table 7 before and after the clinical test. Where the skin condition of the volunteer is improved by three grades or above after the clinical test, it was rated as excellent. Where the skin condition is improved by one or two grades after the clinical test, it was rated as good. The evaluation results are given in Table 15 below.

TABLE 15
Results of clinical test for preparation containing pine pollen itself

| Effects | Persons |
|---|---|
| Excellent | 4 |
| Good | 4 |
| Unchanged | 2 |
| Worse | 0 |
| Side effect | 0 |
| Total | 10 |

From the results given in Table 15, it was found that pine pollen itself may also be used for the prevention and treatment of acne, according to a property and state of a preparation containing pine pollen itself. However, it was found that using pine pollen extract rather than using pine pollen itself is preferred in view of feelings as well as antibacterial effect against the acne bacterium.

### INDUSTRIAL APPLICABILITY

As described above, it was found that pine pollen or pine pollen extract used in the practice of the present invention exhibits an excellent antibacterial effect against the acne bacterium Propionibacterium acnes and an excellent comedo reducing effect, and does not cause any side effects, even when it is used for a lengthy period of time. Therefore, pine pollen or pine pollen extract can be used as an active ingredient of various skin external preparations for preventing and treating acne.

While there have been illustrated and described what are considered to be preferred specific embodiments of the present invention, it will be understood by those skilled in the art that the present invention is not limited to the specific embodiments thereof, and various changes and modifications and equivalents may be substituted for elements thereof without departing from the true scope of the present invention.

1. A skin external preparation containing pine pollen or pine pollen extract in an amount effective in preventing and treating acne.

2. The skin external preparation of claim 1, which is in the form of a treating agent for pharmaceutical application.

3. The skin external preparation of claim 1, which is in the form of a cosmetic selected from the group consisting of a toilet water, a cream, a pack, a lotion, a foundation, a sun screen and a baby powder.

4. The skin external preparation of claim 1, which is in the form of a cleansing agent selected from the group consisting of a soap, a cleansing foam, a hair shampoo and a body shampoo.

5. The skin external preparation of claim 1, in which the pine pollen or the pine pollen extract is contained in an amount ranging from about 0.05% by weight to about 50% by weight, based on the total weight of the skin external preparation.

6. The skin external preparation of claim 5, in which the pine pollen or the pine pollen extract is contained in an amount ranging from about 1% by weight to about 20% by weight, based on the total weight of the skin external preparation.

7. The skin external preparation of claim 6, in which the pine pollen or the pine pollen extract is contained in an amount ranging from about 2% by weight to about 10% by weight, based on the total weight of the skin external preparation.
